(19) United States
(12) Patent Application Publication
(10) Pub. No.: US 2023/0040249 A1
(43) Pub. Date: Feb. 9, 2023

(54) Method of Demineralizing Bone with an Ionic Solution

(71) Applicants: SeaSpine, Inc., Carlsbad, CA (US); Newsouth Innovations Pty Limited, UNSW Sydney NSW (AU)

(72) Inventors: Frank Vizesi, Carlsbad, CA (US); William Robert Walsh, Maroubra (AU)

(21) Appl. No.: 17/876,993

(22) Filed: Jul. 29, 2022

Related U.S. Application Data
(60) Provisional application No. 63/228,411, filed on Aug. 2, 2021.

(51) Int. Cl. A61K 35/32; A61K 38/18; A61K 38/30; A61K 47/02

(52) U.S. Cl. CPC A61K 35/32; A61K 38/1875; A61K 38/1866; A61K 38/1841; A61K 38/30; A61K 47/02

(57) ABSTRACT

A method of producing an osteoinductive composition for in vivo use, in which the method includes obtaining bone tissue; cleaning the bone tissue with a plurality of washes; milling, grinding, and/or cutting the cleaned bone tissue into a plurality of bone pieces or fibers of a desired size; and demineralizing the plurality of bone pieces or fibers of a desired size to expose one or more native bone proteins, with demineralizing including combining the plurality of bone pieces or fibers in a reaction vessel with an ionic acid solution, soaking the plurality of bone pieces or fibers in the ionic acid solution, and exposing the one or more native proteins in the bone pieces or fibers.

### CROSS-REFERENCE TO RELATED APPLICATIONS

This application claims the benefit of U.S. Provisional Application No. 63/228,411, filed Aug. 2, 2021, the contents of which is incorporated by reference in its entirety.

### FIELD OF THE INVENTION

The present disclosure provides a method of producing an osteoinductive composition by demineralizing bone tissue with an acid solution containing salt. The described method, and variants thereof, yield an osteoinductive composition that has a concentration of one or more bone proteins comparatively greater than bone tissue soaked in deionized water acid solution. This disclosure also provides for producing an osteoinductive composition in which the freeze-dried composition may be optionally processed into a shaped material.

### BACKGROUND

Conventionally, the mechanism of bone formation of a demineralized bone matrix (hereinafter “DBM”) is the osteoinductive potential of the DBM, which is the release of native bone proteins such as bone morphogenetic proteins (hereinafter “BMPs”) from the collagenous matrix of the bone tissue. These native growth factors are normally in the bone tissue, but are substantially unavailable for release from the matrix because they are trapped within the mineral component of the bone. Osteoinductivity is achieved by removing the mineral component of the bone (e.g. demineralizing), which is typically done through an acid washing step. This demineralization leaves behind a collagen matrix and the various growth factors that may now be readily released in vivo.

Osteoinductivity of the DBM may be affected by the demineralization process and the extent of demineralization. For example, there may be a “sweet spot” for demineralization above 0% and below about 5% residual calcium. The extent of demineralization, and therefore the osteoinductive potential, may be affected in many ways including the strength of the acid, the ratio of acid to bone, temperature, stirring/agitation, time, and potentially the size/shape of the bone particles.

Ionic content and concentration have complex effects on protein stability by modifying conformational stability and solubility. Ions binding to proteins may crosslink charged protein surfaces and lead to stabilization of the protein's native state but may also affect large dipole moments present in peptide bonds and destabilizing the native state. Depending on the competing strength of these interactions, native proteins may either be stabilized or destabilized. The net effect of salt on protein stability is a balance of multiple mechanisms by which ions interact with protein molecules individually and protein-protein interactions. As pH also determines type, total, and distribution of charges in a protein the ionic effects of salts are also strongly pH dependent. See Chi et al., Pharma. Res., Vol. 20, No. 9, 1325-1336, September 2003.

Methods of improving the osteoinductive potential of the DBM are desired, as the greater the release of native bone proteins, the faster bone formation and healing may be after implantation of DBM within a patient.

### SUMMARY

In one embodiment, a method of producing an osteoinductive composition is described, with the method comprising: (a) obtaining bone tissue; (b) cleaning the bone tissue with one or more washes; (c) demineralizing the bone tissue to expose one or more native bone proteins, wherein demineralizing in step (c) comprises: (i) combining the bone tissue with an acid solution containing salt having a concentration between about 0.5M to 10M; (ii) soaking the bone tissue in the acid solution containing salt; and (iii) exposing the one or more native bone proteins in the bone tissue; and yielding an osteoinductive composition having a bone protein concentration comparatively greater than bone tissue soaked in deionized water acid solution.

In an embodiment, a method of producing an osteoinductive composition as described herein further provides the bone tissue comprises cortical bone, cancellous bone, or a combination thereof.

In another embodiment, a method of producing an osteoinductive composition as described herein further provides the bone tissue comprises whole or fractional bone, bone pieces, bone chips, bone blocks, bone fibrils, bone fiber, bone particles, bone strips, bone powder, or any combination thereof.

In a further embodiment, a method of producing an osteoinductive composition as described herein further provides the acid solution containing salt concentration is between about 0.5 to 2M, or between about 1 to 2M.

In yet another embodiment, a method of producing an osteoinductive composition as described herein further provides the one or more native bone proteins include, but are not limited to, bone morphogenetic protein-2 (BMP-2), bone morphogenetic protein-3 (BMP-3), bone morphogenetic protein-4 (BMP-4), bone morphogenetic protein-5 (BMP-5), bone morphogenetic protein-6 (BMP-6), bone morphogenetic protein-7 (BMP-7), bone morphogenetic protein-8 (BMP-8), bone morphogenetic protein-9 (BMP-9), bone morphogenetic protein-10 (BMP-10), bone morphogenetic protein-12 (BMP-12), bone morphogenetic protein-13 (BMP-13), bone morphogenetic protein-14 (BMP-14), bone morphogenetic protein-15 (BMP-15), collagen and collagen-type proteins, non-collagenous bone proteins, transforming growth factor beta (TGF-beta), insulin-like growth factor (IGF), vascular endothelial growth factor (VEGF), or any combination thereof.

In still another embodiment, a method of producing an osteoinductive composition as described herein further provides the salt includes, but is not limited to, sodium chloride, sodium sulfate, magnesium sulfate, calcium chloride, calcium nitrate, potassium chloride, barium chloride, calcium phosphate, any other salt capable of producing an ionic solution with acid, or any combination thereof.

In another embodiment, a method of producing an osteoinductive composition as described herein further provides the acid solution may comprise one or more components including, but not limited to, sodium chloride, sodium sulfate, magnesium sulfate, calcium chloride, calcium nitrate, calcium phosphate, potassium chloride, barium chloride, glucose, or any combination thereof.

In a further embodiment, a method of producing an osteoinductive composition as described herein further provides the acid solution containing salt comprises an acid including, but not limited to, hydrochloric acid, hydrofluoric acid, acetic acid, citric acid, lactic acid, malic acid, any other acid known to demineralize bone, or any combination thereof.

In another embodiment, a method of producing an osteoinductive composition as described herein further comprises (d) processing the osteoinductive composition to produce a putty, gel, block, strip, sheet, wafer, shaped implant, or a combination thereof.

In a further embodiment, a method of producing an osteoinductive composition as described herein further provides the osteoinductive composition further comprises one or more components including, but not limited to, carboxymethylcellulose, chitosan, collagen, hone mineral, glycerol, hyaluronic acid, hydroxyapatite, sodium alginate, tricalcium phosphate, biphasic calcium phosphate, calcium sulfate, biological glass, poloxamer 407, DB M, a reverse phase medium, a cross-linking agent, or any combination thereof.

In another embodiment, a method of producing an osteoinductive composition comprises: (a) obtaining bone tissue; (b) cleaning the bone tissue with one or more washes; (c) demineralizing the bone tissue to expose one or more native bone proteins, wherein demineralizing in step (c) comprises: (i) combining the bone tissue with an acid solution containing salt having a concentration between about 0.5M to 10M; (ii) soaking the bone tissue in the acid solution containing salt; and (iii) exposing the one or more native bone proteins in the bone tissue; and (d) freeze-drying the demineralized bone tissue produced in step (c); yielding an osteoinductive composition having a bone protein concentration comparatively greater than bone tissue soaked in deionized water acid solution.

In a further embodiment, a method of producing an osteoinductive composition as described herein further provides the bone tissue comprises cortical bone, cancellous bone, or a combination thereof.

In yet another embodiment, a method of producing an osteoinductive composition as described herein further provides the bone tissue comprises whole or fractional bone, bone pieces, bone chips, bone blocks, bone fibrils, bone fiber, bone particles, bone strips, bone powder, or any combination thereof.

In still another embodiment, a method of producing an osteoinductive composition as described herein further provides the acid solution containing salt concentration is between about 0.5 to 2M, or about 1 to 2M.

In a further embodiment, a method of producing an osteoinductive composition as described herein further provides the one or more native bone proteins include, but are not limited to, bone morphogenetic protein-2 (BMP-2), bone morphogenetic protein-3 (BMP-3), bone morphogenetic protein-4 (BMP-4), bone morphogenetic protein-5 (BMP-5), bone morphogenetic protein-6 (BMP-6), bone morphogenetic protein-7 (BMP-7), bone morphogenetic protein-8 (BMP-8), bone morphogenetic protein-9 (BMP-9), bone morphogenetic protein-10 (BMP-10), bone morphogenetic protein-12 (BMP-12), bone morphogenetic protein-13 (BMP-13), bone morphogenetic protein-14 (BMP-14), bone morphogenetic protein-15 (BMP-15), collagen and collagen-type proteins, non-collagenous bone proteins, transforming growth factor beta (TGF-beta), insulin-like growth factor (IGF), vascular endothelial growth factor (VEGF), or any combination thereof.

In another embodiment, a method of producing an osteoinductive composition as described herein further provides the salt includes, but is not limited to, sodium chloride, sodium sulfate, magnesium sulfate, calcium chloride, calcium nitrate, potassium chloride, barium chloride, calcium phosphate, or any other salt capable of producing an ionic solution with acid, or any combination thereof.

In a further embodiment, a method of producing an osteoinductive composition as described herein further provides the acid solution further comprises one or more components including, but not limited to, sodium chloride, sodium sulfate, magnesium sulfate, calcium chloride, calcium nitrate, potassium chloride, barium chloride, calcium phosphate, glucose, or any combination thereof.

In another embodiment, a method of producing an osteoinductive composition as described herein further provides the acid solution containing salt comprises an acid including, but not limited to, hydrochloric acid, hydrofluoric acid, acetic acid, citric acid, lactic acid, malic acid, or any combination thereof.

In yet another embodiment, a method of producing an osteoinductive composition as described herein further provides (e) processing the osteoinductive composition to produce a putty, gel, block, strip, sheet, wafer, shaped implant, or a combination thereof.

In still another embodiment, a method of producing an osteoinductive composition as described herein further provides the osteoinductive composition further comprises one or more components including, but not limited to, carboxymethylcellulose, chitosan, collagen, bone mineral, glycerol, hyaluronic acid, hydroxyapatite sodium alginate, tricalcium phosphate, bipliasic calcium phosphate, calcium sulfate, biological glass, poloxamer 407, DBM, a reverse phase medium, a cross-linking agent, or any combination thereof.

In one aspect, a method of producing an osteoinductive composition is disclosed herein, the method comprising: (a) obtaining bone tissue; (b) cleaning the bone tissue with a plurality of washes; (c) milling or grinding the bone tissue into a plurality of bone pieces; and (d) demineralizing the plurality of bone pieces to expose one or more native bone proteins, wherein demineralizing in step (d) comprises: (i) combining the plurality of bone pieces with an ionic acid solution; (ii) soaking the plurality of bone pieces in the ionic acid solution; and (iii) exposing the one or more native proteins in the bone pieces; to yield an osteoinductive composition.

In one or more embodiments, a method of producing an osteoinductive composition as disclosed herein further comprises in step (ii) rinsing the bone pieces with deionized water. In some embodiments, a method of producing an osteoinductive composition as disclosed herein further comprises in step (ii) rinsing the bone pieces with a salt solution.

In one or more embodiments, a method of producing an osteoinductive composition as disclosed herein includes a salt solution selected from the group consisting of: sodium chloride, sodium sulfate, magnesium sulfate, calcium nitrate, potassium chloride, barium chloride, calcium phosphate, and any combination thereof. In some embodiments, a method of producing an osteoinductive composition as disclosed herein further comprises an ionic acid solution comprising one or more components selected from the group consisting of: sodium chloride, sodium sulfate, magnesium sulfate, calcium nitrate, potassium chloride, barium chloride, calcium phosphate, glucose, and any combination thereof. In some embodiments, the ionic concentration of the ionic acid solution is greater than about 0.5M. In some embodiments, the ionic acid solution concentration is between about 0.5M and about 10M. In one or more embodiments, the ionic acid solution concentration is between about 1M and about 2M.

In one or more embodiments, a method of producing an osteoinductive composition as disclosed herein includes an ionic acid solution comprising an acid selected from the group consisting of: hydrochloric acid, hydrofluoric acid, acetic acid, citric acid, acetic acid, lactic acid, malic acid, or any combination thereof.

In one or more embodiments, a method of producing an osteoinductive composition as disclosed herein includes one or more native proteins including, but not limited to: bone morphogenetic protein-2 (BMP-2), bone morphogenetic protein-4 (BMP-4), bone morphogenetic protein-6 (BMP-6), bone morphogenetic protein-7 (BMP-7), transforming growth factor beta (TGF-beta), insulin-like growth factor (IGF), vascular endothelial growth factor (VEGF), or any combination thereof.

In one or more embodiments, a method of producing an osteoinductive composition as disclosed herein includes an ionic acid solution comprising 0.5M hydrochloric acid. In some embodiments, the ionic acid solution comprises 0.5M hydrochloric acid, wherein the amount of hydrochloric acid is determined by about 40 ml hydrochloric acid per gram of the bone pieces.

In another aspect, a method of producing an osteoinductive composition is disclosed herein, the method comprising: (a) obtaining bone tissue; (b) cleaning the bone tissue with a plurality of washes; (c) milling or grinding the bone tissue into a plurality of bone pieces; and (d) demineralizing the plurality of bone pieces to expose one or more native bone proteins, wherein demineralizing in step (d) comprises: (i) combining the plurality of bone pieces with an ionic acid solution; (ii) soaking the plurality of bone pieces in the ionic acid solution; and (iii) exposing the one or more native proteins in the bone pieces; to yield an osteoinductive composition, wherein step (c) occurs before step (b). In some embodiments of the method as described herein, step (d) occurs before step (c). In some embodiments of the method as described herein, soaking in step (ii) comprises a period of about 60 to about 90 minutes.

In another aspect, a method of producing an osteoinductive composition is disclosed herein, the method comprising: (a) obtaining bone tissue; (b) cleaning the bone tissue with a plurality of washes; (c) milling or grinding the bone tissue into a plurality of bone pieces; and (d) demineralizing the plurality of bone pieces to expose one or more native bone proteins, wherein demineralizing in step (d) comprises: (i) combining the plurality of bone pieces with an ionic acid solution; (ii) soaking the plurality of bone pieces in the ionic acid solution; (iii) exposing the one or more native proteins in the bone pieces; and (iv) freeze-drying the demineralized bone pieces; to yield an osteoinductive composition. In some embodiments of the method as described herein, step (ii) further comprises rinsing the bone pieces with deionized water. In some embodiments of the method as described herein, step (ii) further comprises rinsing the bone pieces with a salt solution.

In one or more embodiments, a method of producing an osteoinductive composition as disclosed herein includes a salt solution selected from the group consisting of: sodium chloride, sodium sulfate, magnesium sulfate, calcium nitrate, potassium chloride, barium chloride, calcium phosphate, and any combination thereof. In some embodiments, a method of producing an osteoinductive composition as disclosed herein further comprises an ionic acid solution comprising one or more components selected from the group consisting of: sodium chloride, sodium sulfate, magnesium sulfate, calcium nitrate, potassium chloride, barium chloride, calcium phosphate, glucose, and any combination thereof. In some embodiments, the ionic concentration of the ionic acid solution is greater than about 0.5M. In some embodiments, the ionic acid solution concentration is between about 0.5M and about 10M. In one or more embodiments, the ionic acid solution concentration is between about 1M and about 2M.

In one or more embodiments, a method of producing an osteoinductive composition as disclosed herein includes an ionic acid solution comprising an acid selected from the group consisting of: hydrochloric acid, hydrofluoric acid, acetic acid, citric acid, acetic acid, lactic acid, malic acid, and any combination thereof.

In one or more embodiments, a method of producing an osteoinductive composition as disclosed herein includes one or more native proteins selected from the group consisting of: bone morphogenetic protein-2 (BMP-2), bone morphogenetic protein-4 (BMP-4), bone morphogenetic protein-6 (BMP-6), bone morphogenetic protein-7 (BMP-7), transforming growth factor beta (TGF-beta), insulin-like growth factor (IGF), vascular endothelial growth factor (VEGF), and any combination thereof.

In one or more embodiments, a method of producing an osteoinductive composition as disclosed herein includes an ionic acid solution comprising 0.5M hydrochloric acid, wherein the amount of hydrochloric acid is determined by about 40 ml hydrochloric acid per gram of the bone pieces.

In another aspect, a method of producing an osteoinductive composition is disclosed herein, the method comprising: (a) obtaining bone tissue; (b) cleaning the bone tissue with a plurality of washes; (c) milling or grinding the bone tissue into a plurality of bone pieces; and (d) demineralizing the plurality of bone pieces to expose one or more native bone proteins, wherein demineralizing in step (d) comprises: (i) combining the plurality of bone pieces with an ionic acid solution; (ii) soaking the plurality of bone pieces in the ionic acid solution; (iii) exposing the one or more native proteins in the bone pieces; and (iv) freeze-drying the demineralized bone pieces; to yield an osteoinductive composition, wherein step (c) occurs before step (b). In some embodiments of the method as disclosed herein, step (d) occurs before step (c). In some embodiments of the method as disclosed herein, soaking in step (ii) comprises a period of about 60 to about 90 minutes.

In another aspect, a method of producing an osteoinductive composition is disclosed herein, the method comprising: (a) obtaining bone tissue; (b) cleaning the bone tissue with a plurality of washes; (c) milling or grinding the bone tissue into a plurality of bone pieces; and (d) demineralizing the plurality of bone pieces to expose one or more native bone proteins, wherein demineralizing in step (d) comprises: (i) combining the plurality of bone pieces with an ionic acid solution; (ii) soaking the plurality of bone pieces in the ionic acid solution; (iii) exposing the one or more native proteins in the bone pieces; and (iv) freeze-drying the demineralized bone pieces; to yield an osteoinductive composition having a moisture content of less than about 6% by weight. In some embodiments of the method as described herein, step (ii) further comprises rinsing the bone pieces with deionized water. In some embodiments of the method as described herein, step (ii) further comprises rinsing the bone pieces with a salt solution.

In one or more embodiments, a method of producing an osteoinductive composition as disclosed herein includes a salt solution selected from the group consisting of: sodium chloride, sodium sulfate, magnesium sulfate, calcium nitrate, potassium chloride, barium chloride, calcium phosphate, and any combination thereof. In some embodiments of the method as described herein, the ionic acid solution comprises one or more components selected from the group consisting of: sodium chloride, sodium sulfate, magnesium sulfate, calcium nitrate, potassium chloride, barium chloride, calcium phosphate, glucose, and any combination thereof. In some embodiments of the method as described herein, the ionic concentration of the ionic acid solution is greater than about 0.5M. In some embodiments, the ionic acid solution concentration is between about 0.5M and about 10M. In one or more embodiments, the ionic acid solution concentration is between about 1M and about 2M.

In one or more embodiments, a method of producing an osteoinductive composition as disclosed herein includes an ionic acid solution comprising an acid selected from the group consisting of: hydrochloric acid, hydrofluoric acid, acetic acid, citric acid, acetic acid, lactic acid, malic acid, and any combination thereof.

In one or more embodiments, a method of producing an osteoinductive composition as disclosed herein includes one or more native proteins selected from the group consisting of: bone morphogenetic protein-2 (BMP-2), bone morphogenetic protein-4 (BMP-4), bone morphogenetic protein-6 (BMP-6), bone morphogenetic protein-7 (BMP-7), transforming growth factor beta (TGF-beta), insulin-like growth factor (IGF), vascular endothelial growth factor (VEGF), and any combination thereof.

In one or more embodiments, a method of producing an osteoinductive composition as disclosed herein includes an ionic acid solution comprising 0.5M hydrochloric acid, wherein the amount of hydrochloric acid is determined by about 40 ml hydrochloric acid per gram of the bone pieces.

In another aspect, a method of producing an osteoinductive composition is disclosed herein, the method comprising: (a) obtaining bone tissue; (b) cleaning the bone tissue with a plurality of washes; (c) milling or grinding the bone tissue into a plurality of bone pieces; and (d) demineralizing the plurality of bone pieces to expose one or more native bone proteins, wherein demineralizing in step (d) comprises: (i) combining the plurality of bone pieces with an ionic acid solution; (ii) soaking the plurality of bone pieces in the ionic acid solution; (iii) exposing the one or more native proteins in the bone pieces; and (iv) freeze-drying the demineralized bone pieces; to yield an osteoinductive composition having a moisture content of less than about 6% by weight, wherein step (c) occurs before step (b). In some embodiments of the method as described herein, step (d) occurs before step (c). In some embodiments of the method as described herein, soaking in step (ii) comprises a period of about 60 to about 90 minutes.

In another aspect, a method of producing an osteoinductive composition is disclosed herein, the method comprising: (a) obtaining cortical bone tissue; (b) cleaning the bone tissue with a plurality of washes; (c) milling or grinding the bone tissue into a plurality of bone pieces; and (d) demineralizing the plurality of bone pieces to expose one or more native bone proteins, wherein demineralizing in step (d) comprises: (i) combining the plurality of bone pieces with an ionic acid solution; (ii) soaking the plurality of bone pieces in the ionic acid solution; and (iii) exposing the one or more native proteins in the bone pieces; to yield an osteoinductive composition. In some embodiments of the method as described herein, step (ii) further comprises rinsing the bone pieces with deionized water. In some embodiments of the method as described herein, step (ii) further comprises rinsing the bone pieces with a salt solution.

In one or more embodiments, a method of producing an osteoinductive composition as disclosed herein includes a salt solution selected from the group consisting of: sodium chloride, sodium sulfate, magnesium sulfate, calcium nitrate, potassium chloride, barium chloride, calcium phosphate, and any combination thereof. In some embodiments of the method as disclosed herein, the ionic acid solution comprises one or more components selected from the group consisting of: sodium chloride, sodium sulfate, magnesium sulfate, calcium nitrate, potassium chloride, barium chloride, calcium phosphate, glucose, and any combination thereof. In some embodiments of the method as disclosed herein, the ionic concentration of the ionic acid solution is greater than about 0.5M. In some embodiments, the ionic acid solution concentration is between about 0.5M and about 10M. In one or more embodiments, the ionic acid solution concentration is between about 1M and about 2M.

In one or more embodiments, a method of producing an osteoinductive composition as disclosed herein includes an ionic acid solution comprising an acid selected from the group consisting of: hydrochloric acid, hydrofluoric acid, acetic acid, citric acid, acetic acid, lactic acid, malic acid, and any combination thereof.

In one or more embodiments, a method of producing an osteoinductive composition as disclosed herein includes one or more native proteins selected from the group consisting of: bone morphogenetic protein-2 (BMP-2), bone morphogenetic protein-4 (BMP-4), bone morphogenetic protein-6 (BMP-6), bone morphogenetic protein-7 (BMP-7), transforming growth factor beta (TGF-beta), insulin-like growth factor (IGF), vascular endothelial growth factor (VEGF), and any combination thereof.

In one or more embodiments, a method of producing an osteoinductive composition as disclosed herein includes an ionic acid solution comprising 0.5M hydrochloric acid, wherein the ionic acid solution comprises 0.5M hydrochloric acid, and wherein the amount of hydrochloric acid is determined by about 40 ml hydrochloric acid per gram of the bone pieces.

In another aspect, a method of producing an osteoinductive composition is disclosed herein, the method comprising: (a) obtaining cortical bone tissue; (b) cleaning the bone tissue with a plurality of washes; (c) milling or grinding the bone tissue into a plurality of bone pieces; and (d) demineralizing the plurality of bone pieces to expose one or more native bone proteins, wherein demineralizing in step (d) comprises: (i) combining the plurality of bone pieces with an ionic acid solution; (ii) soaking the plurality of bone pieces in the ionic acid solution; and (iii) exposing the one or more native proteins in the bone pieces; to yield an osteoinductive composition, wherein step (c) occurs before step (b). In some embodiments of the method as disclosed herein, step (d) occurs before step (c). In some embodiments of the method as disclosed herein, soaking in step (ii) comprises a period of about 60 to about 90 minutes.

In another aspect, a method of producing an osteoinductive composition is disclosed herein, the method comprising: (a) obtaining cortical bone tissue; (b) cleaning the bone tissue with a plurality of washes; (c) milling or grinding the bone tissue into a plurality of bone pieces; and (d) demineralizing the plurality of bone pieces to expose one or more native bone proteins, wherein demineralizing in step (d) comprises: (i) combining the plurality of bone pieces with an ionic acid solution; (ii) soaking the plurality of bone pieces in the ionic acid solution; (iii) exposing the one or more native proteins in the bone pieces; and (iv) freeze-drying the demineralized bone pieces; to yield an osteoinductive composition. In some embodiments of the method as described herein, step (ii) further comprises rinsing the bone pieces with deionized water. In some embodiments of the method as described herein, step (ii) further comprises rinsing the bone pieces with a salt solution.

In one or more embodiments, a method of producing an osteoinductive composition as disclosed herein includes a salt solution selected from the group consisting of: sodium chloride, sodium sulfate, magnesium sulfate, calcium nitrate, potassium chloride, barium chloride, calcium phosphate, and any combination thereof. In some embodiments of the method as disclosed herein, the ionic acid solution comprises one or more components selected from the group consisting of: sodium chloride, sodium sulfate, magnesium sulfate, calcium nitrate, potassium chloride, barium chloride, calcium phosphate, glucose, and any combination thereof. In some embodiments of the method as disclosed herein, the ionic concentration of the ionic acid solution is greater than about 0.5M. In some embodiments, the ionic acid solution concentration is between about 0.5M and about 10M. In one or more embodiments, the ionic acid solution concentration is between about 1M and about 2M.

In one or more embodiments, a method of producing an osteoinductive composition as disclosed herein includes an ionic acid solution comprising an acid selected from the group consisting of: hydrochloric acid, hydrofluoric acid, acetic acid, citric acid, acetic acid, lactic acid, malic acid, and any combination thereof.

In one or more embodiments, a method of producing an osteoinductive composition as disclosed herein includes one or more native proteins selected from the group consisting of: bone morphogenetic protein-2 (BMP-2), bone morphogenetic protein-4 (BMP-4), bone morphogenetic protein-6 (BMP-6), bone morphogenetic protein-7 (BMP-7), transforming growth factor beta (TGF-beta), insulin-like growth factor (IGF), vascular endothelial growth factor (VEGF), and any combination thereof.

In one or more embodiments, a method of producing an osteoinductive composition as disclosed herein includes an ionic acid solution comprising 0.5M hydrochloric acid, wherein the amount of hydrochloric acid is determined by about 40 ml hydrochloric acid per gram of the bone pieces.

In another aspect, a method of producing an osteoinductive composition is disclosed herein, the method comprising: (a) obtaining cortical bone tissue; (b) cleaning the bone tissue with a plurality of washes; (c) milling or grinding the bone tissue into a plurality of bone pieces; and (d) demineralizing the plurality of bone pieces to expose one or more native bone proteins, wherein demineralizing in step (d) comprises: (i) combining the plurality of bone pieces with an ionic acid solution; (ii) soaking the plurality of bone pieces in the ionic acid solution; (iii) exposing the one or more native proteins in the bone pieces; and (iv) freeze-drying the demineralized bone pieces; to yield an osteoinductive composition, wherein step (c) occurs before step (b). In some embodiments of the method as described herein, step (d) occurs before step (c). In some embodiments of the method as described herein, soaking in step (ii) comprises a period of about 60 to about 90 minutes.

In another aspect, a method of producing an osteoinductive composition is disclosed herein, the method comprising: (a) obtaining cortical bone tissue; (b) cleaning the bone tissue with a plurality of washes; (c) milling or grinding the bone tissue into a plurality of bone pieces; and (d) demineralizing the plurality of bone pieces to expose one or more native bone proteins, wherein demineralizing in step (d) comprises: (i) combining the plurality of bone pieces with an ionic acid solution; (ii) soaking the plurality of bone pieces in the ionic acid solution; (iii) exposing the one or more native proteins in the bone pieces; and (iv) freeze-drying the demineralized bone pieces; to yield an osteoinductive composition having a moisture content of less than about 6% by weight. In some embodiments of the method as described herein, step (ii) further comprises rinsing the bone pieces with deionized water. In some embodiments of the method as described herein, step (ii) further comprises rinsing the bone pieces with a salt solution.

In one or more embodiments, a method of producing an osteoinductive composition as disclosed herein includes a salt solution selected from the group consisting of: sodium chloride, sodium sulfate, magnesium sulfate, calcium nitrate, potassium chloride, barium chloride, calcium phosphate, and any combination thereof. In some embodiments of the method as disclosed herein, the ionic acid solution comprises one or more components selected from the group consisting of: sodium chloride, sodium sulfate, magnesium sulfate, calcium nitrate, potassium chloride, barium chloride, calcium phosphate, glucose, and any combination thereof. In some embodiments of the method as disclosed herein, the ionic concentration of the ionic acid solution is greater than about 0.5M. In some embodiments, the ionic acid solution concentration is between about 0.5M and about 10M. In one or more embodiments, the ionic acid solution concentration is between about 1M and about 2M.

In one or more embodiments, a method of producing an osteoinductive composition as disclosed herein includes an ionic acid solution comprising an acid selected from the group consisting of: hydrochloric acid, hydrofluoric acid, acetic acid, citric acid, acetic acid, lactic acid, malic acid, and any combination thereof.

In one or more embodiments, a method of producing an osteoinductive composition as disclosed herein includes one or more native proteins selected from the group consisting of: bone morphogenetic protein-2 (BMP-2), bone morphogenetic protein-4 (BMP-4), bone morphogenetic protein-6 (BMP-6), bone morphogenetic protein-7 (BMP-7), transforming growth factor beta (TGF-beta), insulin-like growth factor (IGF), vascular endothelial growth factor (VEGF), and any combination thereof.

In one or more embodiments, a method of producing an osteoinductive composition as disclosed herein includes an ionic acid solution comprising 0.5M hydrochloric acid, wherein the amount of hydrochloric acid is determined by about 40 ml hydrochloric acid per gram of the bone pieces.

In another aspect, a method of producing an osteoinductive composition is disclosed herein, the method comprising: (a) obtaining cortical bone tissue; (b) cleaning the bone tissue with a plurality of washes; (c) milling or grinding the bone tissue into a plurality of bone pieces; and (d) demineralizing the plurality of bone pieces to expose one or more native bone proteins, wherein demineralizing in step (d) comprises: (i) combining the plurality of bone pieces with an ionic acid solution; (ii) soaking the plurality of bone pieces in the ionic acid solution; (iii) exposing the one or more native proteins in the bone pieces; and (iv) freeze-drying the demineralized bone pieces; to yield an osteoinductive composition having a moisture content of less than about 6% by weight, wherein step (c) occurs before step (b). In some embodiments of the method as described herein, step (d) occurs before step (c). In some embodiments of the method as described herein, soaking in step (ii) comprises a period of about 60 to about 90 minutes.

In another aspect, a method of producing an osteoinductive composition is disclosed herein, the method comprising: (a) obtaining cancellous bone tissue; (b) cleaning the bone tissue with a plurality of washes; (c) milling or grinding the bone tissue into a plurality of bone pieces; and (d) demineralizing the plurality of bone pieces to expose one or more native bone proteins, wherein demineralizing in step (d) comprises: (i) combining the plurality of bone pieces with an ionic acid solution; (ii) soaking the plurality of bone pieces in the ionic acid solution; and (iii) exposing the one or more native proteins in the bone pieces; to yield an osteoinductive composition. In some embodiments of the method as described herein, step (ii) further comprises rinsing the bone pieces with deionized water. In some embodiments of the method as described herein, step (ii) further comprises rinsing the bone pieces with a salt solution.

In one or more embodiments, a method of producing an osteoinductive composition as disclosed herein includes a salt solution selected from the group consisting of: sodium chloride, sodium sulfate, magnesium sulfate, calcium nitrate, potassium chloride, barium chloride, calcium phosphate, and any combination thereof. In some embodiments of the method as disclosed herein, the ionic acid solution comprises one or more components selected from the group consisting of: sodium chloride, sodium sulfate, magnesium sulfate, calcium nitrate, potassium chloride, barium chloride, calcium phosphate, glucose, and any combination thereof. In some embodiments of the method as disclosed herein, the ionic concentration of the ionic acid solution is greater than about 0.5M. In some embodiments, the ionic acid solution concentration is between about 0.5M and about 10M. In one or more embodiments, the ionic acid solution concentration is between about 1M and about 2M.

In one or more embodiments, a method of producing an osteoinductive composition as disclosed herein includes an ionic acid solution comprising an acid selected from the group consisting of: hydrochloric acid, hydrofluoric acid, acetic acid, citric acid, acetic acid, lactic acid, malic acid, and any combination thereof.

In one or more embodiments, a method of producing an osteoinductive composition as disclosed herein includes one or more native proteins are selected from the group consisting of: bone morphogenetic protein-2 (BMP-2), bone morphogenetic protein-4 (BMP-4), bone morphogenetic protein-6 (BMP-6), bone morphogenetic protein-7 (BMP-7), transforming growth factor beta (TGF-beta), insulin-like growth factor (IGF), vascular endothelial growth factor (VEGF), and any combination thereof.

In one or more embodiments, a method of producing an osteoinductive composition as disclosed herein includes an ionic acid solution comprising 0.5M hydrochloric acid, wherein the amount of hydrochloric acid is determined by about 40 ml hydrochloric acid per gram of the bone pieces.

In another aspect, a method of producing an osteoinductive composition is disclosed herein, the method comprising: (a) obtaining cancellous bone tissue; (b) cleaning the bone tissue with a plurality of washes; (c) milling or grinding the bone tissue into a plurality of bone pieces; and (d) demineralizing the plurality of bone pieces to expose one or more native bone proteins, wherein demineralizing in step (d) comprises: (i) combining the plurality of bone pieces with an ionic acid solution; (ii) soaking the plurality of bone pieces in the ionic acid solution; and (iii) exposing the one or more native proteins in the bone pieces; to yield an osteoinductive composition, wherein step (c) occurs before step (b). In some embodiments of the method as described herein, step (d) occurs before step (c). In some embodiments of the method as described herein, soaking in step (ii) comprises a period of about 60 to about 90 minutes.

In another aspect, a method of producing an osteoinductive composition is disclosed herein, the method comprising: (a) obtaining cancellous bone tissue; (b) cleaning the bone tissue with a plurality of washes; (c) milling or grinding the bone tissue into a plurality of bone pieces; and (d) demineralizing the plurality of bone pieces to expose one or more native bone proteins, wherein demineralizing in step (d) comprises: (i) combining the plurality of bone pieces with an ionic acid solution; (ii) soaking the plurality of bone pieces in the ionic acid solution; (iii) exposing the one or more native proteins in the bone pieces; and (iv) freeze-drying the demineralized bone pieces; to yield an osteoinductive composition. In some embodiments of the method as described herein, step (ii) further comprises rinsing the bone pieces with deionized water. In some embodiments of the method as described herein, step (ii) further comprises rinsing the bone pieces with a salt solution.

In one or more embodiments, a method of producing an osteoinductive composition as disclosed herein includes a salt solution selected from the group consisting of: sodium chloride, sodium sulfate, magnesium sulfate, calcium nitrate, potassium chloride, barium chloride, calcium phosphate, and any combination thereof. In some embodiments of the method as disclosed herein, the ionic acid solution comprises one or more components selected from the group consisting of: sodium chloride, sodium sulfate, magnesium sulfate, calcium nitrate, potassium chloride, barium chloride, calcium phosphate, glucose, and any combination thereof.

In one or more embodiments, a method of producing an osteoinductive composition as disclosed herein includes an ionic acid solution comprising 0.5M hydrochloric acid, wherein the amount of hydrochloric acid is determined by about 40 ml hydrochloric acid per gram of the bone pieces.

In one or more embodiments, a method of producing an osteoinductive composition as disclosed herein includes an ionic concentration of the ionic acid solution greater than about 0.5M. In one or more embodiments, the ionic acid solution concentration is between about 0.5M and about 10M. In one or more embodiments, the ionic acid solution concentration is between about 1M and about 2M.

In one or more embodiments, a method of producing an osteoinductive composition as disclosed herein includes an ionic acid solution comprising an acid selected from the group consisting of: hydrochloric acid, hydrofluoric acid, acetic acid, citric acid, acetic acid, lactic acid, malic acid, and any combination thereof.

In one or more embodiments, a method of producing an osteoinductive composition as disclosed herein includes one or more native proteins are selected from the group consisting of: bone morphogenetic protein-2 (BMP-2), bone morphogenetic protein-4 (BMP-4), bone morphogenetic protein-6 (BMP-6), bone morphogenetic protein-7 (BMP-7), transforming growth factor beta (TGF-beta), insulin-like growth factor (IGF), vascular endothelial growth factor (VEGF), and any combination thereof.

In one or more embodiments, a method of producing an osteoinductive composition as disclosed herein includes an ionic acid solution comprising 0.5M hydrochloric acid, wherein the amount of hydrochloric acid is determined by about 40 ml hydrochloric acid per gram of the bone pieces.

In another aspect, a method of producing an osteoinductive composition is disclosed herein, the method comprising: (a) obtaining cancellous bone tissue; (b) cleaning the bone tissue with a plurality of washes; (c) milling or grinding the bone tissue into a plurality of bone pieces; and (d) demineralizing the plurality of bone pieces to expose one or more native bone proteins, wherein demineralizing in step (d) comprises: (i) combining the plurality of bone pieces with an ionic acid solution; (ii) soaking the plurality of bone pieces in the ionic acid solution; (iii) exposing the one or more native proteins in the bone pieces; and (iv) freeze-drying the demineralized bone pieces; to yield an osteoinductive composition, wherein step (c) occurs before step (b). In some embodiments of the method as described herein, step (d) occurs before step (c). In some embodiments of the method as described herein, soaking in step (ii) comprises a period of about 60 to about 90 minutes.

In another aspect, a method of producing an osteoinductive composition is disclosed herein, the method comprising: (a) obtaining cancellous bone tissue; (b) cleaning the bone tissue with a plurality of washes; (c) milling or grinding the bone tissue into a plurality of bone pieces; and (d) demineralizing the plurality of bone pieces to expose one or more native bone proteins, wherein demineralizing in step (d) comprises: (i) combining the plurality of bone pieces with an ionic acid solution; (ii) soaking the plurality of bone pieces in the ionic acid solution; (iii) exposing the one or more native proteins in the bone pieces; and (iv) freeze-drying the demineralized bone pieces; to yield an osteoinductive composition having a moisture content of less than about 6% by weight. In some embodiments of the method as described herein, step (ii) further comprises rinsing the bone pieces with deionized water. In some embodiments of the method as described herein, step (ii) further comprises rinsing the bone pieces with a salt solution.

In one or more embodiments, a method of producing an osteoinductive composition as disclosed herein includes a salt solution selected from the group consisting of: sodium chloride, sodium sulfate, magnesium sulfate, calcium nitrate, potassium chloride, barium chloride, calcium phosphate, and any combination thereof. In some embodiments of the method as disclosed herein, the ionic acid solution comprises one or more components selected from the group consisting of: sodium chloride, sodium sulfate, magnesium sulfate, calcium nitrate, potassium chloride, barium chloride, calcium phosphate, glucose, and any combination thereof. In some embodiments of the method as disclosed herein, the ionic concentration of the ionic acid solution is greater than about 0.5M. In some embodiments, the ionic acid solution concentration is between about 0.5M and about 10M. In one or more embodiments, the ionic acid solution concentration is between about 1M and about 2M.

In one or more embodiments, a method of producing an osteoinductive composition as disclosed herein includes an ionic acid solution comprising an acid selected from the group consisting of: hydrochloric acid, hydrofluoric acid, acetic acid, citric acid, acetic acid, lactic acid, malic acid, and any combination thereof.

In one or more embodiments, a method of producing an osteoinductive composition as disclosed herein includes one or more native proteins are selected from the group consisting of: bone morphogenetic protein-2 (BMP-2), bone morphogenetic protein-4 (BMP-4), bone morphogenetic protein-6 (BMP-6), bone morphogenetic protein-7 (BMP-7), transforming growth factor beta (TGF-beta), insulin-like growth factor (IGF), vascular endothelial growth factor (VEGF), and any combination thereof.

In one or more embodiments, a method of producing an osteoinductive composition as disclosed herein includes an ionic acid solution comprising 0.5M hydrochloric acid, wherein the amount of hydrochloric acid is determined by about 40 ml hydrochloric acid per gram of the bone pieces.

In another aspect, a method of producing an osteoinductive composition is disclosed herein, the method comprising: (a) obtaining cancellous bone tissue; (b) cleaning the bone tissue with a plurality of washes; (c) milling or grinding the bone tissue into a plurality of bone pieces; and (d) demineralizing the plurality of bone pieces to expose one or more native bone proteins, wherein demineralizing in step (d) comprises: (i) combining the plurality of bone pieces with an ionic acid solution; (ii) soaking the plurality of bone pieces in the ionic acid solution; (iii) exposing the one or more native proteins in the bone pieces; and (iv) freeze-drying the demineralized bone pieces; to yield an osteoinductive composition having a moisture content of less than about 6% by weight, wherein step (c) occurs before step (b). In some embodiments of the method as described herein, step (d) occurs before step (c). In some embodiments of the method as described herein, soaking in step (ii) comprises a period of about 60 to about 90 minutes.

In another aspect, a method of producing an osteoinductive composition is disclosed herein, the method comprising: (a) obtaining bone tissue; (b) cleaning the bone tissue with a plurality of washes; (c) cutting the bone tissue into a plurality of bone fibers; and (d) demineralizing the plurality of bone fibers to expose one or more native bone proteins, wherein demineralizing in step (d) comprises: (i) combining the plurality of bone fibers with an ionic acid solution; (ii) soaking the plurality of bone fibers in the ionic acid solution; and (iii) exposing the one or more native proteins in the bone fibers; to yield an osteoinductive composition. In some embodiments of the method as described herein, step (ii) further comprises rinsing the bone pieces with deionized water. In some embodiments of the method as described herein, step (ii) further comprises rinsing the bone pieces with a salt solution.

In one or more embodiments, a method of producing an osteoinductive composition as disclosed herein includes a salt solution selected from the group consisting of: sodium chloride, sodium sulfate, magnesium sulfate, calcium nitrate, potassium chloride, barium chloride, calcium phosphate, and any combination thereof. In some embodiments of the method as disclosed herein, the ionic acid solution comprises one or more components selected from the group consisting of: sodium chloride, sodium sulfate, magnesium sulfate, calcium nitrate, potassium chloride, barium chloride, calcium phosphate, glucose, and any combination thereof.

In one or more embodiments, a method of producing an osteoinductive composition as disclosed herein includes an ionic acid solution comprising 0.5M hydrochloric acid, wherein the amount of hydrochloric acid is determined by about 40 ml hydrochloric acid per gram of the bone pieces.

In one or more embodiments, a method of producing an osteoinductive composition as disclosed herein includes an ionic concentration of the ionic acid solution greater than about 0.5M. In one or more embodiments, the ionic acid solution concentration is between about 0.5M and about 10M. In one or more embodiments, the ionic acid solution concentration is between about 1M and about 2M.

In one or more embodiments, a method of producing an osteoinductive composition as disclosed herein includes an ionic acid solution comprising an acid selected from the group consisting of: hydrochloric acid, hydrofluoric acid, acetic acid, citric acid, acetic acid, lactic acid, malic acid, and any combination thereof.

In one or more embodiments, a method of producing an osteoinductive composition as disclosed herein includes one or more native proteins are selected from the group consisting of: bone morphogenetic protein-2 (BMP-2), bone morphogenetic protein-4 (BMP-4), bone morphogenetic protein-6 (BMP-6), bone morphogenetic protein-7 (BMP-7), transforming growth factor beta (TGF-beta), insulin-like growth factor (IGF), vascular endothelial growth factor (VEGF), and any combination thereof.

In one or more embodiments, a method of producing an osteoinductive composition as disclosed herein includes an ionic acid solution comprising 0.5M hydrochloric acid, wherein the ionic acid solution comprises 0.5M hydrochloric acid, and wherein the amount of hydrochloric acid is determined by about 40 ml hydrochloric acid per gram of the bone fibers.

In another aspect, a method of producing an osteoinductive composition is disclosed herein, the method comprising: (a) obtaining bone tissue; (b) cleaning the bone tissue with a plurality of washes; (c) cutting the bone tissue into a plurality of bone fibers; and (d) demineralizing the plurality of bone fibers to expose one or more native bone proteins, wherein demineralizing in step (d) comprises: (i) combining the plurality of bone fibers with an ionic acid solution; (ii) soaking the plurality of bone fibers in the ionic acid solution; and (iii) exposing the one or more native proteins in the bone fibers; to yield an osteoinductive composition, wherein step (c) occurs before step (b). In some embodiments of the method as described herein, step (d) occurs before step (c). In some embodiments of the method as described herein, soaking in step (ii) comprises a period of about 60 to about 90 minutes.

In another aspect, a method of producing an osteoinductive composition is disclosed herein, the method comprising: (a) obtaining bone tissue; (b) cleaning the bone tissue with a plurality of washes; (c) cutting the bone tissue into a plurality of bone fibers; and (d) demineralizing the plurality of bone fibers to expose one or more native bone proteins, wherein demineralizing in step (d) comprises: (i) combining the plurality of bone fibers with an ionic acid solution; (ii) soaking the plurality of bone fibers in the ionic acid solution; (iii) exposing the one or more native proteins in the bone fibers; and (iv) freeze-drying the demineralized bone fibers; to yield an osteoinductive composition. In some embodiments of the method as described herein, step (ii) further comprises rinsing the bone fibers with deionized water. In some embodiments of the method as described herein, step (ii) further comprises rinsing the bone fibers with a salt solution.

In one or more embodiments, a method of producing an osteoinductive composition as disclosed herein includes a salt solution selected from the group consisting of: sodium chloride, sodium sulfate, magnesium sulfate, calcium nitrate, potassium chloride, barium chloride, calcium phosphate, and any combination thereof. In some embodiments of the method as disclosed herein, the ionic acid solution comprises one or more components selected from the group consisting of: sodium chloride, sodium sulfate, magnesium sulfate, calcium nitrate, potassium chloride, barium chloride, calcium phosphate, glucose, and any combination thereof. In some embodiments of the method as disclosed herein, the ionic concentration of the ionic acid solution is greater than about 0.5M. In some embodiments, the ionic acid solution concentration is between about 0.5M and about 10M. In one or more embodiments, the ionic acid solution concentration is between about 1M and about 2M.

In one or more embodiments, a method of producing an osteoinductive composition as disclosed herein includes an ionic acid solution comprising an acid selected from the group consisting of: hydrochloric acid, hydrofluoric acid, acetic acid, citric acid, acetic acid, lactic acid, malic acid, and any combination thereof.

In one or more embodiments, a method of producing an osteoinductive composition as disclosed herein includes one or more native proteins are selected from the group consisting of: bone morphogenetic protein-2 (BMP-2), bone morphogenetic protein-4 (BMP-4), bone morphogenetic protein-6 (BMP-6), bone morphogenetic protein-7 (BMP-7), transforming growth factor beta (TGF-beta), insulin-like growth factor (IGF), vascular endothelial growth factor (VEGF), and any combination thereof.

In one or more embodiments, a method of producing an osteoinductive composition as disclosed herein includes an ionic acid solution comprising 0.5M hydrochloric acid, and wherein the amount of hydrochloric acid is determined by about 40 ml hydrochloric acid per gram of the bone fibers.

In another aspect, a method of producing an osteoinductive composition is disclosed herein, the method comprising: (a) obtaining bone tissue; (b) cleaning the bone tissue with a plurality of washes; (c) cutting the bone tissue into a plurality of bone fibers; and (d) demineralizing the plurality of bone fibers to expose one or more native bone proteins, wherein demineralizing in step (d) comprises: (i) combining the plurality of bone fibers with an ionic acid solution; (ii) soaking the plurality of bone fibers in the ionic acid solution; (iii) exposing the one or more native proteins in the bone fibers; and (iv) freeze-drying the demineralized bone particles; to yield an osteoinductive composition having a moisture content of less than about 6% by weight.

In some embodiments of the method as described herein, step (ii) further comprises rinsing the bone fibers with deionized water. In some embodiments of the method as described herein, step (ii) further comprises rinsing the bone fibers with a salt solution.

In one or more embodiments, a method of producing an osteoinductive composition as disclosed herein includes a salt solution selected from the group consisting of: sodium chloride, sodium sulfate, magnesium sulfate, calcium nitrate, potassium chloride, barium chloride, calcium phosphate, and any combination thereof. In some embodiments, of the method as disclosed herein, the ionic acid solution comprises one or more components selected from the group consisting of: sodium chloride, sodium sulfate, magnesium sulfate, calcium nitrate, potassium chloride, barium chloride, calcium phosphate, glucose, and any combination thereof. In some embodiments, of the method as disclosed herein, the ionic concentration of the ionic acid solution is greater than about 0.5M. In some embodiments, the ionic acid solution concentration is between about 0.5M and about 10M. In one or more embodiments, the ionic acid solution concentration is between about 1M and about 2M.

In one or more embodiments, a method of producing an osteoinductive composition as disclosed herein includes an ionic acid solution comprising an acid selected from the group consisting of: hydrochloric acid, hydrofluoric acid, acetic acid, citric acid, acetic acid, lactic acid, malic acid, and any combination thereof.

In one or more embodiments, a method of producing an osteoinductive composition as disclosed herein includes one or more native proteins are selected from the group consisting of: bone morphogenetic protein-2 (BMP-2), bone morphogenetic protein-4 (BMP-4), bone morphogenetic protein-6 (BMP-6), bone morphogenetic protein-7 (BMP-7), transforming growth factor beta (TGF-beta), insulin-like growth factor (IGF), vascular endothelial growth factor (VEGF), and any combination thereof.

In one or more embodiments, a method of producing an osteoinductive composition as disclosed herein includes an ionic acid solution comprising 0.5M hydrochloric acid, wherein the amount of hydrochloric acid is determined by about 40 ml hydrochloric acid per gram of the bone fibers.

In another aspect, a method of producing an osteoinductive composition is disclosed herein, the method comprising: (a) obtaining bone tissue; (b) cleaning the bone tissue with a plurality of washes; (c) cutting the bone tissue into a plurality of bone fibers; and (d) demineralizing the plurality of bone fibers to expose one or more native bone proteins, wherein demineralizing in step (d) comprises: (i) combining the plurality of bone fibers with an ionic acid solution; (ii) soaking the plurality of bone fibers in the ionic acid solution; (iii) exposing the one or more native proteins in the bone fibers; and (iv) freeze-drying the demineralized bone particles; to yield an osteoinductive composition having a moisture content of less than about 6% by weight, wherein step (c) occurs before step (b). In some embodiments of the method as described herein, step (d) occurs before step (c). In some embodiments of the method as described herein, soaking in step (ii) comprises a period of about 60 to about 90 minutes.

In another aspect, a method of producing an osteoinductive composition is disclosed herein, the method comprising: (a) obtaining cortical bone tissue; (b) cleaning the bone tissue with a plurality of washes; (c) cutting the bone tissue into a plurality of bone fibers; and (d) demineralizing the plurality of bone fibers to expose one or more native bone proteins, wherein demineralizing in step (d) comprises: (i) combining the plurality of bone fibers with an ionic acid solution; (ii) soaking the plurality of bone fibers in the ionic acid solution; and (iii) exposing the one or more native proteins in the bone fibers; to yield an osteoinductive composition. In some embodiments of the method as described herein, step (ii) further comprises rinsing the bone fibers with deionized water. In some embodiments of the method as described herein, step (ii) further comprises rinsing the bone fibers with a salt solution.

In one or more embodiments, a method of producing an osteoinductive composition as disclosed herein includes a salt solution selected from the group consisting of: sodium chloride, sodium sulfate, magnesium sulfate, calcium nitrate, potassium chloride, barium chloride, calcium phosphate, and any combination thereof. In some embodiments, of the method as disclosed herein, the ionic acid solution comprises one or more components selected from the group consisting of: sodium chloride, sodium sulfate, magnesium sulfate, calcium nitrate, potassium chloride, barium chloride, calcium phosphate, glucose, and any combination thereof. In some embodiments, of the method as disclosed herein, the ionic concentration of the ionic acid solution is greater than about 0.5M. In some embodiments, the ionic acid solution concentration is between about 0.5M and about 10M. In one or more embodiments, the ionic acid solution concentration is between about 1M and about 2M.

In one or more embodiments, a method of producing an osteoinductive composition as disclosed herein includes an ionic acid solution comprising an acid selected from the group consisting of: hydrochloric acid, hydrofluoric acid, acetic acid, citric acid, acetic acid, lactic acid, malic acid, and any combination thereof.

In one or more embodiments, a method of producing an osteoinductive composition as disclosed herein includes one or more native proteins are selected from the group consisting of: bone morphogenetic protein-2 (BMP-2), bone morphogenetic protein-4 (BMP-4), bone morphogenetic protein-6 (BMP-6), bone morphogenetic protein-7 (BMP-7), transforming growth factor beta (TGF-beta), insulin-like growth factor (IGF), vascular endothelial growth factor (VEGF), and any combination thereof.

In one or more embodiments, a method of producing an osteoinductive composition as disclosed herein includes an ionic acid solution comprising 0.5M hydrochloric acid, wherein the amount of hydrochloric acid is determined by about 40 ml hydrochloric acid per gram of the bone fibers.

In another aspect, a method of producing an osteoinductive composition is disclosed herein, the method comprising: (a) obtaining cortical bone tissue; (b) cleaning the bone tissue with a plurality of washes; (c) cutting the bone tissue into a plurality of bone fibers; and (d) demineralizing the plurality of bone fibers to expose one or more native bone proteins, wherein demineralizing in step (d) comprises: (i) combining the plurality of bone fibers with an ionic acid solution; (ii) soaking the plurality of bone fibers in the ionic acid solution; and (iii) exposing the one or more native proteins in the bone fibers; to yield an osteoinductive composition, wherein step (c) occurs before step (b). In some embodiments of the method as described herein, step (d) occurs before step (c). In some embodiments of the method as described herein, soaking in step (ii) comprises a period of about 60 to about 90 minutes.

In another aspect, a method of producing an osteoinductive composition is disclosed herein, the method comprising: (a) obtaining cortical bone tissue; (b) cleaning the bone tissue with a plurality of washes; (c) cutting the bone tissue into a plurality of bone fibers; and (d) demineralizing the plurality of bone fibers to expose one or more native bone proteins, wherein demineralizing in step (d) comprises: (i) combining the plurality of bone fibers with an ionic acid solution; (ii) soaking the plurality of bone fibers in the ionic acid solution; (iii) exposing the one or more native proteins in the bone fibers; and (iv) freeze-drying the demineralized bone fibers; to yield an osteoinductive composition. In some embodiments of the method as described herein, step (ii) further comprises rinsing the bone fibers with deionized water. In some embodiments of the method as described herein, step (ii) further comprises rinsing the bone fibers with a salt solution.

In one or more embodiments, a method of producing an osteoinductive composition as disclosed herein includes a salt solution selected from the group consisting of: sodium chloride, sodium sulfate, magnesium sulfate, calcium nitrate, potassium chloride, barium chloride, calcium phosphate, and any combination thereof. In some embodiments, of the method as disclosed herein, the ionic acid solution comprises one or more components selected from the group consisting of: sodium chloride, sodium sulfate, magnesium sulfate, calcium nitrate, potassium chloride, barium chloride, calcium phosphate, glucose, and any combination thereof. In some embodiments, of the method as disclosed herein, the ionic concentration of the ionic acid solution is greater than about 0.5M. In some embodiments, the ionic acid solution concentration is between about 0.5M and about 10M. In one or more embodiments, the ionic acid solution concentration is between about 1M and about 2M.

In one or more embodiments, a method of producing an osteoinductive composition as disclosed herein includes an ionic acid solution comprising an acid selected from the group consisting of: hydrochloric acid, hydrofluoric acid, acetic acid, citric acid, acetic acid, lactic acid, malic acid, and any combination thereof.

In one or more embodiments, a method of producing an osteoinductive composition as disclosed herein includes one or more native proteins are selected from the group consisting of: bone morphogenetic protein-2 (BMP-2), bone morphogenetic protein-4 (BMP-4), bone morphogenetic protein-6 (BMP-6), bone morphogenetic protein-7 (BMP-7), transforming growth factor beta (TGF-beta), insulin-like growth factor (IGF), vascular endothelial growth factor (VEGF), and any combination thereof.

In one or more embodiments, a method of producing an osteoinductive composition as disclosed herein includes an ionic acid solution comprising 0.5M hydrochloric acid, wherein the amount of hydrochloric acid is determined by about 40 ml hydrochloric acid per gram of the bone fibers.

In another aspect, a method of producing an osteoinductive composition is disclosed herein, the method comprising: (a) obtaining cortical bone tissue; (b) cleaning the bone tissue with a plurality of washes; (c) cutting the bone tissue into a plurality of bone fibers; and (d) demineralizing the plurality of bone fibers to expose one or more native bone proteins, wherein demineralizing in step (d) comprises: (i) combining the plurality of bone fibers with an ionic acid solution; (ii) soaking the plurality of bone fibers in the ionic acid solution; (iii) exposing the one or more native proteins in the bone fibers; and (iv) freeze-drying the demineralized bone fibers; to yield an osteoinductive composition, wherein step (c) occurs before step (b). In some embodiments of the method as described herein, step (d) occurs before step (c). In some embodiments of the method as described herein, soaking in step (ii) comprises a period of about 60 to about 90 minutes.

In another aspect, a method of producing an osteoinductive composition is disclosed herein, the method comprising: (a) obtaining cortical bone tissue; (b) cleaning the bone tissue with a plurality of washes; (c) cutting the bone tissue into a plurality of bone fibers; and (d) demineralizing the plurality of bone fibers to expose one or more native bone proteins, wherein demineralizing in step (d) comprises: (i) combining the plurality of bone fibers with an ionic acid solution; (ii) soaking the plurality of bone fibers in the ionic acid solution; (iii) exposing the one or more native proteins in the bone fibers; and (iv) freeze-drying the demineralized bone particles; to yield an osteoinductive composition having a moisture content of less than about 6% by weight. In some embodiments of the method as described herein, step (ii) further comprises rinsing the bone fibers with deionized water. In some embodiments of the method as described herein, step (ii) further comprises rinsing the bone fibers with a salt solution.

In one or more embodiments, a method of producing an osteoinductive composition as disclosed herein includes a salt solution selected from the group consisting of: sodium chloride, sodium sulfate, magnesium sulfate, calcium nitrate, potassium chloride, barium chloride, calcium phosphate, and any combination thereof. In some embodiments of the method as disclosed herein, the ionic acid solution comprises one or more components selected from the group consisting of: sodium chloride, sodium sulfate, magnesium sulfate, calcium nitrate, potassium chloride, barium chloride, calcium phosphate, glucose, and any combination thereof. In some embodiments of the method as disclosed herein, the ionic concentration of the ionic acid solution is greater than about 0.5M. In some embodiments, the ionic acid solution concentration is between about 0.5M and about 10M. In one or more embodiments, the ionic acid solution concentration is between about 1M and about 2M.

In one or more embodiments, a method of producing an osteoinductive composition as disclosed herein includes an ionic acid solution comprising an acid selected from the group consisting of: hydrochloric acid, hydrofluoric acid, acetic acid, citric acid, acetic acid, lactic acid, malic acid, and any combination thereof.

In one or more embodiments, a method of producing an osteoinductive composition as disclosed herein includes one or more native proteins are selected from the group consisting of: bone morphogenetic protein-2 (BMP-2), bone morphogenetic protein-4 (BMP-4), bone morphogenetic protein-6 (BMP-6), bone morphogenetic protein-7 (BMP-7), transforming growth factor beta (TGF-beta), insulin-like growth factor (IGF), vascular endothelial growth factor (VEGF), and any combination thereof.

In one or more embodiments, a method of producing an osteoinductive composition as disclosed herein includes an ionic acid solution comprising 0.5M hydrochloric acid, wherein the amount of hydrochloric acid is determined by about 40 ml hydrochloric acid per gram of the bone fibers.

In another aspect, a method of producing an osteoinductive composition is disclosed herein, the method comprising: (a) obtaining cortical bone tissue; (b) cleaning the bone tissue with a plurality of washes; (c) cutting the bone tissue into a plurality of bone fibers; and (d) demineralizing the plurality of bone fibers to expose one or more native bone proteins, wherein demineralizing in step (d) comprises: (i) combining the plurality of bone fibers with an ionic acid solution; (ii) soaking the plurality of bone fibers in the ionic acid solution; (iii) exposing the one or more native proteins in the bone fibers; and (iv) freeze-drying the demineralized bone particles; to yield an osteoinductive composition having a moisture content of less than about 6% by weight, wherein step (c) occurs before step (b). In some embodiments of the method as described herein, step (d) occurs before step (c). In some embodiments of the method as described herein, soaking in step (ii) comprises a period of about 60 to about 90 minutes.

In another aspect, a method of producing an osteoinductive composition is disclosed herein, the method comprising: (a) obtaining cancellous bone tissue; (b) cleaning the bone tissue with a plurality of washes; (c) cutting the bone tissue into a plurality of bone fibers; and (d) demineralizing the plurality of bone fibers to expose one or more native bone proteins, wherein demineralizing in step (d) comprises: (i) combining the plurality of bone fibers with an ionic acid solution; (ii) soaking the plurality of bone fibers in the ionic acid solution; and (iii) exposing the one or more native proteins in the bone fibers; to yield an osteoinductive composition. In some embodiments of the method as described herein, step (ii) further comprises rinsing the bone fibers with deionized water. In some embodiments of the method as described herein, step (ii) further comprises rinsing the bone fibers with a salt solution.

In one or more embodiments, a method of producing an osteoinductive composition as disclosed herein includes a salt solution selected from the group consisting of: sodium chloride, sodium sulfate, magnesium sulfate, calcium nitrate, potassium chloride, barium chloride, calcium phosphate, and any combination thereof. In some embodiments, of the method as disclosed herein, the ionic acid solution comprises one or more components selected from the group consisting of: sodium chloride, sodium sulfate, magnesium sulfate, calcium nitrate, potassium chloride, barium chloride, calcium phosphate, glucose, and any combination thereof. In some embodiments, of the method as disclosed herein, the ionic concentration of the ionic acid solution is greater than about 0.5M. In some embodiments, the ionic acid solution concentration is between about 0.5M and about 10M. In one or more embodiments, the ionic acid solution concentration is between about 1M and about 2M.

In one or more embodiments, a method of producing an osteoinductive composition as disclosed herein includes an ionic acid solution comprising an acid selected from the group consisting of: hydrochloric acid, hydrofluoric acid, acetic acid, citric acid, acetic acid, lactic acid, malic acid, and any combination thereof.

In one or more embodiments, a method of producing an osteoinductive composition as disclosed herein includes one or more native proteins are selected from the group consisting of: bone morphogenetic protein-2 (BMP-2), bone morphogenetic protein-4 (BMP-4), bone morphogenetic protein-6 (BMP-6), bone morphogenetic protein-7 (BMP-7), transforming growth factor beta (TGF-beta), insulin-like growth factor (IGF), vascular endothelial growth factor (VEGF), and any combination thereof.

In one or more embodiments, a method of producing an osteoinductive composition as disclosed herein includes an ionic acid solution comprising 0.5M hydrochloric acid, wherein the amount of hydrochloric acid is determined by about 40 ml hydrochloric acid per gram of the bone fibers.

In another aspect, a method of producing an osteoinductive composition is disclosed herein, the method comprising: (a) obtaining cancellous bone tissue; (b) cleaning the bone tissue with a plurality of washes; (c) cutting the bone tissue into a plurality of bone fibers; and (d) demineralizing the plurality of bone fibers to expose one or more native bone proteins, wherein demineralizing in step (d) comprises: (i) combining the plurality of bone fibers with an ionic acid solution; (ii) soaking the plurality of bone fibers in the ionic acid solution; and (iii) exposing the one or more native proteins in the bone fibers; to yield an osteoinductive composition, wherein step (c) occurs before step (b). In some embodiments of the method as described herein, step (d) occurs before step (c). In some embodiments of the method as described herein, soaking in step (ii) comprises a period of about 60 to about 90 minutes.

In another aspect, a method of producing an osteoinductive composition is disclosed herein, the method comprising: (a) obtaining cancellous bone tissue; (b) cleaning the bone tissue with a plurality of washes; (c) cutting the bone tissue into a plurality of bone fibers; and (d) demineralizing the plurality of bone fibers to expose one or more native bone proteins, wherein demineralizing in step (d) comprises: (i) combining the plurality of bone fibers with an ionic acid solution; (ii) soaking the plurality of bone fibers in the ionic acid solution; (iii) exposing the one or more native proteins in the bone fibers; and (iv) freeze-drying the demineralized bone fibers; to yield an osteoinductive composition. In some embodiments of the method as described herein, step (ii) further comprises rinsing the bone fibers with deionized water. In some embodiments of the method as described herein, step (ii) further comprises rinsing the bone fibers with a salt solution.

In one or more embodiments, a method of producing an osteoinductive composition as disclosed herein includes a salt solution selected from the group consisting of: sodium chloride, sodium sulfate, magnesium sulfate, calcium nitrate, potassium chloride, barium chloride, calcium phosphate, and any combination thereof. In some embodiments, of the method as disclosed herein, the ionic acid solution comprises one or more components selected from the group consisting of: sodium chloride, sodium sulfate, magnesium sulfate, calcium nitrate, potassium chloride, barium chloride, calcium phosphate, glucose, and any combination thereof. In some embodiments, of the method as disclosed herein, the ionic concentration of the ionic acid solution is greater than about 0.5M. In some embodiments, the ionic acid solution concentration is between about 0.5M and about 10M. In one or more embodiments, the ionic acid solution concentration is between about 1M and about 2M.

In one or more embodiments, a method of producing an osteoinductive composition as disclosed herein includes an ionic acid solution comprising an acid selected from the group consisting of: hydrochloric acid, hydrofluoric acid, acetic acid, citric acid, acetic acid, lactic acid, malic acid, and any combination thereof.

In one or more embodiments, a method of producing an osteoinductive composition as disclosed herein includes one or more native proteins are selected from the group consisting of: bone morphogenetic protein-2 (BMP-2), bone morphogenetic protein-4 (BMP-4), bone morphogenetic protein-6 (BMP-6), bone morphogenetic protein-7 (BMP-7), transforming growth factor beta (TGF-beta), insulin-like growth factor (IGF), vascular endothelial growth factor (VEGF), and any combination thereof.

In one or more embodiments, a method of producing an osteoinductive composition as disclosed herein includes an ionic acid solution comprising 0.5M hydrochloric acid, wherein the amount of hydrochloric acid is determined by about 40 ml hydrochloric acid per gram of the bone fibers.

In another aspect, a method of producing an osteoinductive composition is disclosed herein, the method comprising: (a) obtaining cancellous bone tissue; (b) cleaning the bone tissue with a plurality of washes; (c) cutting the bone tissue into a plurality of bone fibers; and (d) demineralizing the plurality of bone fibers to expose one or more native bone proteins, wherein demineralizing in step (d) comprises: (i) combining the plurality of bone fibers with an ionic acid solution; (ii) soaking the plurality of bone fibers in the ionic acid solution; and (iii) exposing the one or more native proteins in the bone fibers; to yield an osteoinductive composition, wherein step (c) occurs before step (b). In some embodiments of the method as described herein, step (d) occurs before step (c). In some embodiments of the method as described herein, soaking in step (ii) comprises a period of about 60 to about 90 minutes.

In another aspect, a method of producing an osteoinductive composition is disclosed herein, the method comprising: (a) obtaining cancellous bone tissue; (b) cleaning the bone tissue with a plurality of washes; (c) cutting the bone tissue into a plurality of bone fibers; and (d) demineralizing the plurality of bone fibers to expose one or more native bone proteins, wherein demineralizing in step (d) comprises: (i) combining the plurality of bone fibers with an ionic acid solution; (ii) soaking the plurality of bone fibers in the ionic acid solution; (iii) exposing the one or more native proteins in the bone fibers; and (iv) freeze-drying the demineralized bone fibers; to yield an osteoinductive composition having a moisture content of less than about 6% by weight. In some embodiments of the method as described herein, step (ii) further comprises rinsing the bone fibers with deionized water. In some embodiments of the method as described herein, step (ii) further comprises rinsing the bone fibers with a salt solution.

In one or more embodiments, a method of producing an osteoinductive composition as disclosed herein includes a salt solution selected from the group consisting of: sodium chloride, sodium sulfate, magnesium sulfate, calcium nitrate, potassium chloride, barium chloride, calcium phosphate, and any combination thereof. In some embodiments, of the method as disclosed herein, the ionic acid solution comprises one or more components selected from the group consisting of: sodium chloride, sodium sulfate, magnesium sulfate, calcium nitrate, potassium chloride, barium chloride, calcium phosphate, glucose, and any combination thereof. In some embodiments, of the method as disclosed herein, the ionic concentration of the ionic acid solution is greater than about 0.5M. In some embodiments, the ionic acid solution concentration is between about 0.5M and about 10M. In one or more embodiments, the ionic acid solution concentration is between about 1M and about 2M.

In one or more embodiments, a method of producing an osteoinductive composition as disclosed herein includes an ionic acid solution comprising an acid selected from the group consisting of: hydrochloric acid, hydrofluoric acid, acetic acid, citric acid, acetic acid, lactic acid, malic acid, and any combination thereof.

In one or more embodiments, a method of producing an osteoinductive composition as disclosed herein includes one or more native proteins are selected from the group consisting of: bone morphogenetic protein-2 (BMP-2), bone morphogenetic protein-4 (BMP-4), bone morphogenetic protein-6 (BMP-6), bone morphogenetic protein-7 (BMP-7), transforming growth factor beta (TGF-beta), insulin-like growth factor (IGF), vascular endothelial growth factor (VEGF), and any combination thereof.

In one or more embodiments, a method of producing an osteoinductive composition as disclosed herein includes an ionic acid solution comprising 0.5M hydrochloric acid, wherein the amount of hydrochloric acid is determined by about 40 ml hydrochloric acid per gram of the bone fibers.

In another aspect, a method of producing an osteoinductive composition is disclosed herein, the method comprising: (a) obtaining cancellous bone tissue; (b) cleaning the bone tissue with a plurality of washes; (c) cutting the bone tissue into a plurality of bone fibers; and (d) demineralizing the plurality of bone fibers to expose one or more native bone proteins, wherein demineralizing in step (d) comprises: (i) combining the plurality of bone fibers with an ionic acid solution; (ii) soaking the plurality of bone fibers in the ionic acid solution; (iii) exposing the one or more native proteins in the bone fibers; and (iv) freeze-drying the demineralized bone fibers; to yield an osteoinductive composition having a moisture content of less than about 6% by weight, wherein step (c) occurs before step (b). In some embodiments of the method as described herein, step (d) occurs before step (c). In some embodiments of the method as described herein, soaking in step (ii) comprises a period of about 60 to about 90 minutes.

### BRIEF DESCRIPTION OF THE FIGURES

FIG. 1 is a flowchart of an exemplary embodiment of a method of demineralizing bone with an ionic solution.

### DETAILED DESCRIPTION

Generally, osteoinductivity is achieved by removing the mineral component of a bone sample via an acid washing step, leaving behind a collagen matrix and a pool of growth factors. Once demineralized, these growth factors may be readily available for release in vivo where the biological affect is realized. Since the osteoinductive growth factors are proteins, and protein structures are delicate and may be influenced by various factors, methods of demineralizing bone to increase osteoinductive potential were evaluated.

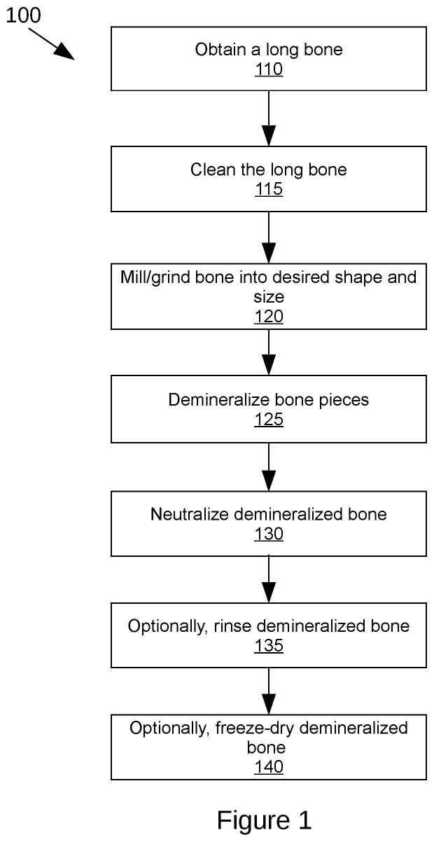

With reference to FIG. 1, an exemplary improved method 100 of demineralizing bone is described. Generally, human long bones are utilized in the preparation of demineralized bone material. In some instances, individual lots of fibers may be processed, where each lot is obtained from a single long bone donor. At block 110 a long bone may be obtained, for example from a tissue bank. Next, at block 115, the obtained long bone may be cleaned. This cleaning may include debridement and then processing with hydrogen peroxide, isopropyl alcohol, and water rinses, although, this is not intended to be limiting, other cleaning protocols known in the art may also be utilized. In some instances, the cortical bone shaft segments may be used, while in other instances, cancellous bone may be used.

At block 120 the cleaned bone segments (either cortical bone or cancellous bone) may then be milled or ground into a plurality of bone pieces of a desired size and shape. For example, in some instances, the cleaned cortical segments may be ground into irregular particles of approximately 150-800 microns. In another example, the cortical particles may be about 1 mm to about 4 mm. In other instances, the cortical segments may be milled into one or more fibers of desired length(s) and width(s), for example on a CNC (Computer Numeric Control) machine using a solid carbide 2-straight flute end mill to achieve fibers.

At block 125, the bone pieces (e.g. particles, fibers, etc.) may be demineralized. In some embodiments, this demineralization may take place in a reaction vessel utilizing 0.5N hydrochloric acid (HCl), where approximately 40 ml HCl/gram of bone is used for approximately 5 to about 9 days. However, this is not intended to be limiting, as the concentration of the acid may vary, for example, depending on the effectiveness of the acid. Additionally, the amount of DBM placed in the acid may vary, depending on the strength of the solution and/or the shape and size of the DBM. For example, in other instances, higher or lower concentrations of HCl may be used, for example a concentration of 0.5M, 0.6M, 1.0M, or any other combination between about 0.5M and about 1.0M may be used in the demineralization step. In other instances, other acids may be used in place of HCl. For example, hydrofluoric acid (HF), acetic acid, citric acid, lactic acid, malic acid, and the like may be used for the demineralization step. As another example, where citric acid is used, about 10 ml to about 50 ml of about 1.0 M to 10.0 M citric acid may be used per gram of DBM. In a further example, about 20 mL of about 2.0 to about 3.0 M citric acid may be used per gram of DBM.

Conventionally, acid demineralization is performed with a deionized (“DI”) water base solution. However, as described herein, an ionic base solution may be used in the preparation of the acid solution. The term “base solution” may also be referred to as a “starting solution”. For example, in some instances, a 1M sodium chloride (NaCl) solution may be used as a base solution for the acid, forming an ionic acid solution. In other instances, the ionic components may vary, for example, sodium sulfate, magnesium sulfate, calcium nitrate, potassium chloride, barium chloride, calcium chloride, calcium phosphate, glucose, a combination thereof, or any other components may be used to generate the ionic base solution. This is not intended to be an exhaustive list, as any known ionic components may be used within the base solution. An increase in ionic concentration may change the ionic strength of the solution, which may also influence protein conformation and result in an increase in osteoinductivity of the resulting demineralized bone.

The demineralization process may expose native proteins in bone. Although the Examples provided herein specifically examine the effect on bone morphogenetic protein 2 (BMP-2), this is not to be construed as limiting. Other native proteins in the bone that may be affected by demineralization with an ionic acid solution may include, but are not limited to: other bone morphogenetic proteins (BMPs, including any of BMPs 1 to 15), for example, BMP-3, BMP-4, BMP-5, BMP-6, and BMP-7, BMP-8, BMP-9, BMP-10, BMP-12, BMP-13, BMP-14, BMP-15, collagen and collagen-type proteins, non-collagenous bone proteins, transforming growth factor beta (TGF-beta), insulin-like growth factor (IGF), and vascular endothelial growth factor (VEGF). Collagen and collagen-type proteins, for example, are described in Ricard-Blum S. The collagen family. Cold Spring Harb Perspect Biol. 2011 Jan. 1; 3(1):a004978.doi: 10.1101/cshperspect.a004978. PMID: 21421911; PMCID: PMC3003457. Non-collagenous bone proteins, for example, are described in Morgan S, Poundarik A A, Vashishth D. Do Non-collagenous Proteins Affect Skeletal Mechanical Properties? Calcif Tissue Int. 2015 September; 97(3):281-91. doi: 0.1007/s00223-015-0016-3. Epub 2015 Jun. 6. PMID: 26048282; PMID4527887.

At block 130, the demineralized bone pieces may be neutralized, for example, by rinsing and/or buffering. The term “bone pieces” as used herein includes bone particles, bone fibers, bone fibrils, bone powder, bone fragments, bone segments, bone sections, bone chunks, and the like. The term “DBM” as used herein also includes demineralized bone particles, demineralized bone fibers, demineralized bone fibrils, demineralized bone powder, demineralized bone fragments, demineralized bone segments, demineralized bone sections, demineralized bone chunks, and the like. In some embodiments, the rinsing may be with reverse osmosis (RO), DI water, or NaCl solution, and the bone pieces may be rinsed about 2 to 3 times, although other multiples of rinsing cycles may be used. In some embodiments, the demineralized bone pieces may be buffered with a 0.1M sodium phosphate buffer (pH 6.9). Optionally, at block 135, the neutralized demineralized bone pieces may be subjected to a final rinse, including multiple final rinses, which may be, for example, either water or a solution of NaCl.

Finally, at block 140, the neutralized and buffered demineralized bone pieces may be subjected to a freeze-drying cycle in order to achieve a moisture content of less than about 6% by weight. As the Examples herein demonstrate, the use of an ionic acid solution in the demineralization of the bone may affect osteoinduction. Also, as shown in the various Examples, DBM particles demonstrated improved osteoinductive ability as measured by native protein content.

The resulting DBM particles may be used in order to increase the bone forming activity by optimizing the demineralization method though use of an ionic acid solution. The resulting demineralized bone may be used in any way a standard demineralized bone matrix may be used, in any bone location, and in any bone graft procedure. For example, the resulting demineralized bone may be used as a bone void filler, a bone graft including a substitute for a bone defect, and may be used in spinal implants procedures, dental implant procedures, bone fusion, and the like.

The methods described herein and illustrated in FIG. 1 are merely exemplary as the order of the operations is not intended to be strictly defined. For example, in some instances, the bone may be cleaned prior to being cut (as illustrated in FIG. 1); in other instances, the bone may be cut and then cleaned. Similarly, the demineralization step could occur prior to the bone being cleaned; furthermore, the bone could also be cleaned following demineralization. One or more additional rinsing steps may also occur before and/or after any enumerated step in FIG. 1 (i.e., before and/or after block 110, 115, 120, 125, 130, 135, and/or 140), or any combination thereof.

In another embodiment, if an acid medium is used, the pH of the osteoinductive composition may be adjusted to be between about 0.5 and about 5.5, or may be between about 0.5 and about 3.5.

The osteoinductive composition may be partially or fully dried to remove excess water. The drying may occur by various means. In an embodiment, the drying occurs by, lyophilization (freeze drying). In another embodiment, the drying is substantially complete, such that most of the water is removed (greater than 95%), and a relatively dry product remains. Alternatively, the lyophilization or other drying process may be arrested at some time prior to completion.

One or more biologically active ingredients may be added to the resulting composition. These active ingredients may or may not be related to the connective tissue repair capabilities of the composition. Suitable active ingredients include DB M containing residual, endogenous bone morphogenetic proteins and related proteins such as cartilage derived morphogenetic proteins (CDMPs). Other active ingredients may be added to the composition, including bone-derived materials such as cortical or cancellous bone chips and bone mineral, osteogenic chemicals (e.g. L-arginine), osteogenic peptides (e.g. OSA), osteogenic growth factors (e.g. transforming growth factor-beta [TGF-β], insulin-like growth factor [IGF], platelet derived growth factor [PDGF], vascular endothelial growth factor [VEGF], fibroblast growth factor [FGF]), and recombinant BMPs (e.g. rBMP-2, rBMP-7), fibronectin, and blood-derived proteins. When added in appropriate combinations, these active ingredients may assist bone repair, cartilage repair, ligament and tendon repair, meniscal repair, and other musculoskeletal applications.

One or more thickening materials may be added to the resulting composition. Any such material may also be an active ingredient or biologically inert. Suitable thickening materials include collagen, insoluble extraction product (Which may or may not contain residual BMPs), bone mineral, hydroxyapatite, tricalcium phosphate, biphasic calcium phosphate, calcium sulfate, biological glasses, and natural or synthetic polymers. Such polymers include poloxamer 407, chitosan, hyaluronic acid, sodium alginate, carboxymethylcellulose, collagen, and related polymers. In an embodiment, DBM, and/or a reverse phase medium may be used as a thickening material with or without added proteins. The reverse phase medium may be an aqueous mixture of Pluronic F127 (BASF Corp) in an amount sufficient to confer a reverse phase property to the composition, such as an approximately 20-40% w/w, or about 23-32% w/w, or about 25% w/w or about 35% w/w mixture of Pluronic F127 and water. Other reverse phase media include aqueous mixtures of derivatives of Plutonic F127.

The biological, physicochemical and biodegradation properties of the composition may be altered by known cross-linking agents such as chemicals (e.g., glutaraldehyde or formaldehyde) or radiation (e.g. gamma or electron beam). In an embodiment, radiation may be used as the cross-linking agent, and electron beam (E-beam) radiation may also be used to irradiate the wet or dry materials at doses between about 5 and about 50 kGray.

In another embodiment, the composition may be injected or inserted at, in, on, or near a bone or chondral defect site. The manner of injection or insertion is not essential, but injection may be via syringe and insertion may be by creating a surgical opening to access the bone or chondral defect site.

In another embodiment, the composition may be mixed with a combination of active and filler or thickening materials such as DBM and insoluble extraction product respectively, and injected or inserted at, in, on, or near a bone or chondral defect site. In an embodiment, the weight to weight (w/w) ratio of DB M to insoluble extraction product is about 3 to 1.

In another embodiment, the dry osteoinductive composition or concentrate may be mixed with aqueous alcohol or other volatile solutions, cast into a desired shape and dried to form a sponge-like material. In one embodiment, a one to six carbon alcohol may be used. In another embodiment, ethanol may be used. In an embodiment, a 1 to 20 percent alcohol by volume solution may be used. In another embodiment, a 4.75 percent ethanol by volume solution may be used. In another embodiment, 20 mg to 200 mg of dry material may be combined with each ML of ethanol. In another embodiment, 50 to 80 mg of dry osteoinductive composition per mL of ethanol may be used, A biologically active ingredient, as discussed above, may also be added. In another embodiment, DBM may be used, Additionally, one or more thickening materials, as discussed above, may also be added, Insoluble extraction product may also be added to this composition. The resulting composition may be cast into a sheet or other shape with or without other added materials. The sheet or other shape is dried. Drying may be achieved by any conventional method, including lyophilization or air-drying. In an embodiment, drying may be by lyophilization.

In another embodiment, the sheet or shape formed with an alcohol solution as described above may be used as or as part of a surgical implant. In an embodiment where a sheet is used, it may be used as a wrap around an area or as a patch inserted into a bone defect site, e.g., insertion into a bone defect, a chondral defect, a spinal fusion cage or a pre-reamed acetabular bed.

In one or more embodiments, the osteoinductive composition may be processed into a putty, a paste, a gel, and/or formed into various shapes. For example, the osteoinductive composition may be shaped into one or more blocks, strips, sheets, or other custom shapes. In one or more embodiments, the shaped material may contain one or more additional components as described herein, or the shaped components may comprise the osteoinductive composition without any additional component(s).

In a further embodiment, the osteoinductive composition may be used to make a sponge-like material that contains demineralized bone particles. The material may be chopped into small pieces of about 0.5 to about 5 cm, or about 1 to about 2 cm. The chopped material may then be combined with aqueous ethanol (approximately 3-10% ethanol, or about 4-5% ethyl alcohol), and mixed until the material is dispersed. DBM particles may then be added at a ratio of about 2-4:1 by weight, or 3:1 by weight and the composition is thoroughly mixed. Then, ethanol may be added to the composition and the mixed composition poured into a container, such as a container that is in the shape of the desired product. The composition may then be refrigerated, frozen and lyophilized to obtain a composition that is substantially free of moisture. The end product may have a sponge-like consistency.

### EXAMPLES

### Example 1

Two separate human cortical bone samples (Sample 1 and Sample 2) were obtained from two separate human donors and cleaned as described above with reference to blocks 110 and 115. Each sample was ground to irregular particles of approximately 300-1000 microns in size, forming a bone powder. Four aliquots of powder from each sample were demineralized in a reaction vessel using 0.5N HCl (made from 4 different base solutions) at approximately 40 ml HCl per gram of bone powder for a time of 120 minutes. The 0.5N HCl demineralizing solution was made with 4 different base solutions: (1) DI water (standard); (2) DI water at 4° C.; (3) 0.0225M NaCl; and (4) 1.0M NaCl. Following treatment with the 0.5N HCl solution, the samples were rinsed with DI water (standard treatment) or 0.0225M NaCl solution or 1M NaCl solution. All samples were buffered with 1× phosphate buffered saline. All samples had a final rinse of DI water or 0.0225M NaCl solution. For samples treated at 4° C., all processing solutions were maintained at 4° C.

Each sample was then electron beam (e-beam) sterilized at 25-35 kGy and tested for its BMP-2 content via an ELISA. 0.5 g of each demineralized sample was added to 6.5 ml of 200 mM HEPES buffer, and samples were hydrated for 2 to 3 hours at 37° C. in a shaking water bath. After hydration, collagenase (at a concentration of 2020 U/mL) was added to each sample and samples were incubated for 15-17 hours, then samples were vortexed, centrifuged and analyzed.

Analysis of the concentration of bone morphogenetic protein-2 (BMP-2) in each sample aliquot was performed utilizing the Quantikine BMP-2 ELISA (R&D Systems®, Minneapolis, Minn.) and a Tecan® plate reader and Magellan software. The results of these analyses are presented in Table 1.

TABLE 1

| | Base Solution for 0.5N | | | |
|---|---|---|---|---|
| | Hydrochloric Acid (HCl) | Average | | Normalized |
| | Demineralization | BMP-2 (ng/g) | Standard | vs DI H2O |
| | Rinse Solution | Triplicate | Deviation | standard |
| Bone | DI H₂O (standard) demineralization | 11.2 | 0.26 | 1.0 |
| Sample 1 | DI H₂O (standard) rinse | | | |
| | 0.0225M NaCl demineralization | 10.7 | 0.25 | 0.96 |
| | 0.0225M NaCl rinse | | | |
| | 1.0M NaCl demineralization | 17.9 | 0.38 | 1.6 |
| | 0.0225M NaCl rinse | | | |
| | DI H₂O at 4° C. demineralization | 12.9 | 0.26 | 1.15 |
| | DI H₂O (standard) rinse | | | |
| Bone | DI H₂O (standard) demineralization | 9.5 | 0.32 | 1.0 |
| Sample 2 | DI H₂O (standard) rinse | | | |
| | 0.0225M NaCl demineralization | 9.4 | 0.53 | 0.99 |
| | 0.0225M NaCl rinse | | | |
| | 1.0M NaCl demineralization | 12.3 | 0.23 | 1.29 |
| | 0.0225M NaCl rinse | | | |
| | DI H₂O at 4° C. demineralization | 11.3 | 0.51 | 1.19 |
| | DI H₂O (standard) rinse | | | |

As illustrated in Table 1, the triplicate average of the standard DI water base HCl demineralization was 11.2 ng/g and 9.5 ng/g of BMP-2 for Bone Sample 1 and 2, respectively. A modest increase in BMP-2 was shown in the cold (4° C.) DI water treatment group as compared to the standard treatment; the triplicate average for the cold (4° C.) DI water base HCl demineralization was to 12.9 ng/g and 11.3 ng/g of BMP-2 for Bone Sample 1 and 2, respectively. The 1.0M NaCl treatment group showed a much larger increase as compared to the standard treatment; the triplicate average for the 1.0M NaCl was to 17.9 ng/g and 12.3 ng/g of BMP-2 for Bone Sample 1 and 2, respectively. Finally, the 0.02255M NaCl treatment group showed no significant increase compared to the standard treatment.

### Example 2

A single human cortical bone sample (Sample 1) was obtained and cleaned as described above with reference to blocks 110 and 115. The bone was then ground to irregular particles of approximately 300-1000 microns in size, forming a bone powder. Eight aliquots of powder were demineralized in a reaction vessel using 0.5N HCl (made from 8 different base solutions) at approximately 40 ml HCl per gram of bone powder for a time of 120 minutes. Eight different base solutions and post-demineralization rinse combinations were analyzed: (1) DI water (standard) base solution and rinse; (2) 0.15M NaCl base solution and 0.0225M NaCl rinse; (3) 0.5M NaCl base solution and 0.0225M NaCl rinse; (4) 1.0M NaCl base solution and 0.0225M NaCl rinse; (5) 1.0M NaCl base solution and 0.15M NaCl rinse; (6) 2.0M NaCl base solution and 0.0225M NaCl rinse; (7) DI water base solution at 4° C. and standard DI water rinse; and (8) 1.0M NaCl base solution at 4° C. and 0.0225M NaCl rinse. All samples were buffered with 1× Phosphate Buffered Saline. For samples treated with 0.5N HCl at 4° C., all processing solutions were maintained at 4° C.

Each sample was then electron beam (e-beam) sterilized at 25-35 kGy and tested for their BMP-2 content via an ELISA assay. Then, 0.5 g of each demineralized sample was added to 6.5 ml of 200 mM HEPES buffer, and samples were hydrated for 2 to 3 hours at 37° C. in a shaking water bath. After hydration, collagenase (at a concentration of 2020 U/mL) was added to each sample and samples were incubated for 15-17 hours; samples were then vortexed and analyzed.

Analysis of the concentration of bone morphogenetic protein-2 (BMP-2) in each sample aliquot was performed utilizing the Quantikine BMP-2 ELISA (R&D Systems®, Minneapolis, Minn.) and a Tecan® plate reader and Magellan software. The results of these analyses are presented in Table 2.

TABLE 2

| Base Solution for 0.5N Hydrochloric Acid | | | |
|---|---|---|---|
| (HCl) Demineralization | Average BMP-2 | Standard | Normalized vs DI |
| Rinse Solution | (ng/g) Triplicate | Deviation | H2O standard |
| DI H2O (standard) demineralization | 10 | 0.71 | 1.0 |
| DI H₂O (standard) rinse | | | |
| 0.15M NaCl demineralization | 10.2 | 0.76 | 1.02 |
| 0.0225M final rinse | | | |
| 0.5M NaCl demineralization | 10.6 | 2.43 | 1.06 |
| 0.0225M final rinse | | | |
| 1M NaCl demineralization | 14.3 | 0.14 | 1.43 |
| 0.0225M final rinse | | | |
| 1M NaCl demineralization | 12.7 | 0.46 | 1.27 |
| 0.15M final rinse | | | |
| 2M NaCl demineralization | 14.6 | 1.14 | 1.46 |
| 0.0225M final rinse | | | |
| DI H₂O at 4° C. demineralization | 9.1 | 0.24 | 0.91 |
| DI H₂O (standard) rinse | | | |
| 1M NaCl at 4° C. demineralization | 13.3 | 1.16 | 1.33 |
| 0.0225M final rinse | | | |

As illustrated in Table 2, as compared to the DI water standard treatment, an increase in bone morphogenetic protein-2 was found when the base solution for the HCl demineralizing treated was the 1M or 2M NaCl concentrations (regardless of temperature or final rinse solution). There was not a marked increase in BMP-2 with cold DI water alone, nor with 0.15M or 0.5M NaCl.

### Example 3

Six human cortical bone samples (Samples 1-6) were obtained and cleaned as described above with reference to blocks 110 and 115 in FIG. 1. The bone was then milled to fibers of approximately 30-50 mm in length. Each cortical bone sample was split into 2 aliquots and then demineralized in a reaction vessel using 0.5N HCl (made from 2 different solutions) at approximately 40 ml HCl per gram of cortical bone for a time of 6 minutes. Two different base solutions and post-demineralization rinse combinations were analyzed: (1) DI water (standard) base solution and rinse; and (2) 1.0M NaCl base solution and 0.0225M NaCl rinse. All samples were buffered with 1× Phosphate Buffered Saline. Each sample was then electron beam (e-beam) sterilized at 25-35 kGy and evaluated for osteoinductivity in triplicate via the rat muscle pouch model at 28 days. Each sample was hydrated with saline and measured to 0.2cc prior to implantation; samples were implanted into intermuscular pockets in the caudal musculature of the rear limbs. The implants remained in the animals for 28 days, after which the implants were analyzed via histology for bone formation and given an osteoinductivity (OI) score that corresponded to the percent positive, as defined by the area of new bone forming elements divided by the total implant area. The results of the study are provided in Table 3.

TABLE 3

| | | Highest New Bone Forming Area (%) | |
|---|---|---|---|
| | | DI H₂O (standard) | 1M NaCl |
| Human Cortical Bone | | demineralization | demineralization |
| Sample | | DI H₂O (standard) rinse | 0.0225M final rinse |
| Sample #1 | a. | 61% | 45% |
| | b. | 45% | 52% |
| | c. | 57% | 65% |
| Sample #2 | a. | 7% | 26% |
| | b. | 14% | 23% |
| | c. | 40% | 33% |
| Sample #3 | a. | 5% | 54% |
| | b. | 18% | 25% |
| | c. | 7% | 54% |
| Sample #4 | a. | 6% | 24% |
| | b. | 2% | 18% |
| | c. | 6% | 33% |
| Sample #5 | a. | 2% | 30% |
| | b. | 4% | 31% |
| | c. | 18% | 23% |
| Sample #6 | a | 3% | 3% |
| | b | 1% | 7% |
| | c | 1% | 23% |

As illustrated in Table 3, as compared to the DI water standard treatment, an increase in new bone forming area was found when the base solution for the HCl demineralizing treated was the 1M NaCl.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

When used in this specification and the claims as an adverb rather than a preposition, “about” means “approximately” and comprises the stated value and every value within 10% of that value; in other words, “about 100%” includes 90% and 110% and every value in between.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of producing an osteoinductive composition, the method comprising:
(a) obtaining bone tissue;
(b) cleaning the bone tissue with one or more washes;
(c) demineralizing the bone tissue to expose one or more native bone proteins, wherein demineralizing in step (c) comprises:
(i) combining the bone tissue with an acid solution containing salt having a concentration between about 0.5M to 10M;
(ii) soaking the bone tissue in the acid solution containing salt; and
(iii) exposing the one or more native bone proteins in the bone tissue; and
yielding the osteoinductive composition having a bone protein concentration comparatively greater than bone tissue soaked in deionized water acid solution.

2. The method of claim 1, wherein the bone tissue comprises cortical bone, cancellous bone, or a combination thereof.

3. The method of claim 1, wherein the bone tissue comprises whole or fractional bone, bone pieces, bone chips, bone blocks, bone fibrils, bone fiber, bone particles, bone strips, bone powder, or any combination thereof.

4. The method of claim 1, wherein the acid solution containing salt concentration is between about 0.5 to 2M.

5. The method of claim 1, wherein the acid solution containing salt concentration is between about 1 to 2M.

6. The method of claim 1, wherein the one or more native bone proteins are selected from the group consisting of bone morphogenetic protein-2 (BMP-2), bone morphogenetic protein-3 (BMP-3), bone morphogenetic protein-4 (BMP-4), bone morphogenetic protein-5 (BMP-5), bone morphogenetic protein-6 (BMP-6), bone morphogenetic protein-7 (BMP-7), bone morphogenetic protein-8 (BMP-8), bone morphogenetic protein-9 (BMP-9), bone morphogenetic protein-10 (BMP-10), bone morphogenetic protein-12 (BMP-12), bone morphogenetic protein-13 (BMP-13), bone morphogenetic protein-14 (BMP-14), bone morphogenetic protein-15 (BMP-15), collagen and collagen-type proteins, non-collagenous bone proteins, transforming growth factor beta (TGF-beta), insulin-like growth factor (IGF), vascular endothelial growth factor (VEGF), and any combination thereof.

7. The method of claim 1, wherein the salt is selected from the group consisting of sodium chloride, sodium sulfate, magnesium sulfate, calcium chloride, calcium nitrate, potassium chloride, barium chloride, calcium phosphate, and any combination thereof.

8. The method of claim 1, wherein the acid solution further comprises one or more components selected from the group consisting of sodium chloride, sodium sulfate, magnesium sulfate, calcium chloride, calcium nitrate, calcium phosphate, potassium chloride, barium chloride, glucose, and any combination thereof.

9. The method of claim 1, wherein the acid solution containing salt comprises an acid selected from the group consisting of hydrochloric acid, hydrofluoric acid, acetic acid, citric acid, lactic acid, malic acid, and any combination thereof.

10. The method of claim 1, further comprising (d) processing the osteoinductive composition to produce a putty, gel, block, strip, sheet, wafer, shaped implant, or a combination thereof.

11. The method of claim 10, wherein the osteoinductive composition further comprises carboxymethylcellulose, chitosan, collagen, bone mineral, glycerol, hyaluronic acid, hydroxyapatite, sodium alginate, tricalcium phosphate, biphasic calcium phosphate, calcium sulfate, biological glass, poloxamer 407, DBM, a reverse phase medium, a cross-linking agent, or any combination thereof.

12. A method of producing an osteoinductive composition, the method comprising:
(a) obtaining bone tissue;
(b) cleaning the bone tissue with one or more washes;
(c) demineralizing the bone tissue to expose one or more native bone proteins, wherein demineralizing in step (c) comprises:
(i) combining the bone tissue with an acid solution containing salt having a concentration between about 0.5M to 10M;
(ii) soaking the bone tissue in the acid solution containing salt; and
(iii) exposing the one or more native bone proteins in the bone tissue; and
(d) freeze-drying the demineralized bone tissue produced in step (c);
yielding the osteoinductive composition having a bone protein concentration comparatively greater than bone tissue soaked in deionized water acid solution.

13. The method of claim 12, wherein the bone tissue comprises cortical bone, cancellous bone, or a combination thereof.

14. The method of claim 12, wherein the bone tissue comprises whole or fractional bone, bone pieces, bone chips, bone fibrils, bone fiber, bone particles, bone strips, bone powder, or any combination thereof.

15. The method of claim 12, wherein the acid solution containing salt concentration is between about 0.5 to 2M.

16. The method of claim 12, wherein the acid solution containing salt concentration is between about 1 to 2M.

17. The method of claim 12, wherein the one or more native bone proteins are selected from the group consisting of bone morphogenetic protein-2 (BMP-2), bone morphogenetic protein-3 (BMP-3), bone morphogenetic protein-4 (BMP-4), bone morphogenetic protein-5 (BMP-5), bone morphogenetic protein-6 (BMP-6), bone morphogenetic protein-7 (BMP-7), bone morphogenetic protein-8 (BMP-8), bone morphogenetic protein-9 (BMP-9), bone morphogenetic protein-10 (BMP-10), bone morphogenetic protein-12 (BMP-12), bone morphogenetic protein-13 (BMP-13), bone morphogenetic protein-14 (BMP-14), bone morphogenetic protein-15 (BMP-15), collagen and collagen-type proteins, non-collagenous bone proteins, transforming growth factor beta (TGF-beta), insulin-like growth factor (IGF), vascular endothelial growth factor (VEGF), and any combination thereof.

18. The method of claim 12, wherein the salt is selected from the group consisting of sodium chloride, sodium sulfate, magnesium sulfate, calcium chloride, calcium nitrate, potassium chloride, barium chloride, calcium phosphate, and any combination thereof.

19. The method of claim 12, wherein the acid solution further comprises one or more components selected from the group consisting of sodium chloride, sodium sulfate, magnesium sulfate, calcium chloride, calcium nitrate, potassium chloride, barium chloride, calcium phosphate, glucose, and any combination thereof.

20. The method of claim 12, wherein the acid solution containing salt comprises an acid selected from the group consisting of hydrochloric acid, hydrofluoric acid, acetic acid, citric acid, lactic acid, malic acid, and any combination thereof.

21. The method of claim 12, further comprising (e) processing the osteoinductive composition to produce a putty, gel, block, strip, sheet, wafer, shaped implant, or a combination thereof.

22. The method of claim 21, wherein the osteoinductive composition further comprises carboxymethylcellulose, chitosan, collagen, hone mineral, glycerol, hyaturonie acid, hydroxyapatite, sodium alginate, tricalcium phosphate, biphasic calcium phosphate, calcium sulfate, biological glass, poloxamer 407, DBM, a reverse phase medium a cross-linking agent, or any combination thereof.
